(19) United States
(12) Patent Application Publication
(10) Pub. No.: US 2010/0152832 A1
(43) Pub. Date: Jun. 17, 2010

(54) Apparatus and Methods for Treatment of Aneurysms With Fibrin Derived Peptide B-Beta

(75) Inventor: AYALA HEZI-YAMIT, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/333,782

(22) Filed: Dec. 12, 2008

(51) Int. Cl. A61F 2/06; A61M 37/00

(52) U.S. Cl. 623/1.13; 623/1.42; 604/508

(57) ABSTRACT

Disclosed herein are methods and stent grafts related to the treatment of aneurysms through the local administration of at least one fibrin derived peptide B-beta. The at least one fibrin derived peptide B-beta can be locally administered by one or more of placing them directly onto a stent graft, incorporating them into a coating found on a stent graft, including them in a delivery device that is associated with a stent graft and/or injecting them through delivery and/or injection catheters at or near the time of stent graft deployment.

### FIELD OF THE INVENTION

The present disclosure generally relates to the treatment of aneurysms through the local administration of at least one fibrin derived peptide B-beta. The at least one fibrin derived peptide B-beta can be locally administered by placing them directly onto a stent graft, incorporating them into a coating found on a stent graft, including them in a delivery device that is associated with a stent graft and/or injecting them through delivery and/or injection catheters at or near the time of stent graft deployment.

### BACKGROUND OF THE INVENTION

Aneurysms arise when a thinning, weakening section of an artery wall balloons out and are generally treated when the artery expands to more than 150% of its normal diameter. The most common and deadly of these occur in the aorta, the large blood vessel stretching from the heart to the lower abdomen. A normal aorta is between 1.6 to 2.8 centimeters wide; if an area reaches as wide as 5.5 centimeters, the risk of rupture increases such that surgery is recommended. Aneurysms are asymptomatic and they often burst before the patient reaches the hospital.

Aneurysms are estimated to cause approximately 32,000 deaths each year in the United States. Additionally, aneurysm deaths are suspected of being underreported because sudden unexplained deaths, about 450,000 in the United States alone, are often simply misdiagnosed as heart attacks or strokes while many of them may be due to aneurysms. Aneurysms most often occur in the aorta, the largest artery in the body. Most aortic aneurysms, approximately 15,000/year, involve the abdominal aorta while approximately 2,500 occur in the chest. Cerebral aneurysms occur in the brain and present a more complicated case because they are more difficult to detect and treat, causing approximately 14,000 U.S. deaths per year. Aortic aneurysms are detected by standard ultrasound, computerized tomography (CT) and magnetic resonance imaging (MRI) scans and the increased use of these scanning techniques for other diseases has produced an estimated 200% increase in the diagnosis of intact aortic aneurysms. Approximately 200,000 intact aortic aneurysms are diagnosed each year due to this increased screening alone.

United States surgeons treat approximately 50,000 abdominal aortic aneurysms each year, typically by replacing the abnormal section of vessel with a polymer graft in an open surgical procedure. A less-invasive procedure that has more recently been used is the placement of a stent graft at the aneurysm site. Stent grafts are tubular devices that span the aneurysm site to provide support without replacing a section of the vessel. The stent graft, when placed within a vessel at an aneurysm site, acts as a barrier between blood flow and the weakened wall of a vessel, thereby decreasing pressure on the damaged portion of the vessel. Patients whose multiple medical comorbidities make them very high risk for conventional aneurysm repair can be candidates for stent grafting.

Despite the effectiveness of stent grafting, once the aneurysmal site is bypassed, the aneurysm remains. The aneurysmic tissue can continue to degenerate such that the aneurysm continues to increase in size due to the continued thinning of the vessel wall. Thus, there is a need in the art to treat aneurysms themselves and/or to slow or stop continued aneurysm growth following stent graft placement. The present disclosure relates to such an advance.

### SUMMARY OF THE INVENTION

The present invention provides methods and stent grafts that can be used to treat aneurysms following stent graft deployment.

In one embodiment, the invention is a method of treating an aneurysm comprising: delivering a stent graft to the site of the aneurysm; deploying the stent graft to span the aneurysm; and locally administering at least one fibrin derived peptide B-beta to the site of the aneurysm.

In another embodiment, described are methods of local administration comprising:
applying the at least one fibrin derived peptide B-beta to the outer surface of the stent graft and/or incorporating the at least one fibrin derived peptide B-beta into a coating on the stent graft.

In another embodiment, described are methods of local administration comprising:
incorporating the at least one fibrin derived peptide B-beta into a coating and placing the coating on the outer surface of the stent graft.

In another embodiment, described are methods of local administration comprising:
attaching a delivery device to the stent graft wherein the delivery device holds and releases at least one fibrin derived peptide B-beta. In another embodiment of the method, the delivery device is a pouch.

In another embodiment, described are methods of local administration comprising:
providing a stent graft with two layers wherein following deployment the first layer is exposed to blood flow and the second layer faces the blood vessel wall and wherein the second layer is semi-permeable; partially adhering the layers together so that pouches are formed; and loading the pouches with at least one fibrin derived peptide B-beta.

In another embodiment, described are methods of local administration comprising: associating the at least one fibrin derived peptide B-beta with a carrier before loading the pouches with the at least one fibrin derived peptide B-beta.

In another embodiment, described are methods of local administration comprising: applying at least one fibrin derived peptide B-beta directly to the outer surface of the stent graft while the stent graft is compressed within a stent deployment catheter.

In another embodiment, described are methods of local administration comprising: administering the at least one fibrin derived peptide B-beta through a delivery catheter and/or an injection catheter.

In another embodiment, described are methods of local administration comprising: the at least one fibrin derived peptide B-beta substantially fill the aneurysm sac.

In another embodiment, described are methods of local administration comprising: the injection catheter is selected from the group comprising a single lumen injection catheter and a multilumen injection catheter.

In another embodiment, described are methods of local administration comprising: administering the at least one fibrin derived peptide B-beta through at least two injection catheters wherein the first and second injection catheters reach the aneurysm through a different route.

The present invention also includes stent grafts that can be used in accordance with the present invention. In one embodiment the invention includes a stent graft comprising at least one fibrin derived peptide B-beta wherein the at least one fibrin derived peptide B-beta are one or more of applied to the outer surface of the stent graft, incorporated within a coating applied to the stent graft or within a delivery device associated with the stent graft.

In another embodiment of the stent grafts, the stent graft comprises at least one fibrin derived peptide B-beta incorporated within a coating applied to the stent graft wherein the coating is biodegradable.

In another embodiment of the stent grafts, the stent graft comprises at least one fibrin derived peptide B-beta incorporated within a coating applied to the stent graft wherein the coating is temperature-sensitive and/or pH-sensitive.

In another embodiment of the stent grafts, the stent graft comprises at least one fibrin derived peptide B-beta incorporated within a coating applied to the stent graft wherein the coating is formulated to be a quick-release coating, a medium-release coating or a slow-release coating.

In another embodiment of the stent grafts, the stent graft comprises at least one fibrin derived peptide B-beta within a delivery device associated with the stent graft and wherein the at least one fibrin derived peptide B-beta are further associated with a carrier.

In another embodiment of the stent grafts, the carrier is selected from the group consisting of a sheet, a slab, a gel, a capsule, capsules, microparticles, nanoparticles, and combinations thereof.

In another embodiment of the stent grafts, the delivery device is a pouch associated with the stent graft. In another embodiment of the stent grafts, the pouch is created by providing a stent graft with two layers wherein following deployment the first layer is exposed to blood flow and the second layer faces the blood vessel wall and wherein the second layer is semi-permeable; and partially adhering the layers together so that one or more pouches are formed.

### BRIEF DESCRIPTION OF THE DRAWINGS

FIG. 1 depicts a fully deployed stent graft with an exterior metal scaffolding as used in an abdominal aortic aneurysm;

FIG. 2 depicts a delivery device associated with a stent graft deployed at an aneurysm site;

FIG. 3a is a side view of a pouch delivery device;

FIG. 3b is a cross-sectional view of a stent graft with a pouch delivery device wrapped around its outer surface;

FIG. 4 illustrates a stent graft delivery catheter adapted to allow coating of the outer wall of a stent graft with at least one fibrin derived peptide B-beta within the delivery catheter;

FIG. 5 illustrates an alternative stent graft delivery catheter adapted to allow coating of the outer wall of a stent graft with at least one fibrin derived peptide B-beta within the delivery catheter;

FIGS. 6a-6c illustrates stent graft deployment with the delivery of at least one fibrin derived peptide B-beta through an injection catheter at the treatment site;

FIGS. 7a-c illustrates stent graft deployment with the delivery of at least one fibrin derived peptide B-beta through injection catheters at the treatment site;

FIG. 8 illustrates an alternate method of delivering at least one fibrin derived peptide B-beta directly into the aneurysm sac after deployment of a stent graft;

FIG. 9 illustrates an alternate method of delivering at least one fibrin derived peptide B-beta directly into the aneurysm sac after deployment of a stent graft; and

FIG. 10 illustrates yet another alternate method of delivering at least one fibrin derived peptide B-beta directly into the aneurysm sac after deployment of a stent graft.

### DETAILED DESCRIPTION OF THE INVENTION

An aneurysm is a swelling, or expansion of a blood vessel and is generally associated with a vessel wall defect. Previous methods to treat aneurysms involved highly invasive surgical procedures where the affected vessel region was removed (or opened) and replaced (or supplemented internally) with a synthetic graft that was sutured in place. However, this procedure was highly invasive and not appropriate for all patients. Historically, patients who were not candidates for this procedure remained untreated and thus at continued risk for sudden death due to aneurysm rupture.

To overcome some of the risks associated with invasive aneurysmal surgeries, stent grafts were developed. Stent grafts can be positioned and deployed using minimally invasive procedures. Essentially, a catheter having a stent graft compressed and fitted into the catheter's distal tip is advanced through an artery to a position spanning the aneurysmal site. The stent graft is then deployed within the vessel lumen juxtaposed to the weakened vessel wall forming an inner liner that insulates the aneurysm from passing blood flow and its resulting hemodynamic forces that can promote stress and rupture. The size and shape of the stent graft is matched to the treatment site's lumen diameter and aneurysm length.

Stent grafts generally comprise a metal scaffolding having a biocompatible graft material lining or covering such as Dacron®, expanded polytetrafluoroethylene, or a fabric-like material woven from a variety of biocompatible polymer fibers. The graft material can be stitched, glued or molded to the scaffold. When a self-expanding stent graft is deployed from the delivery catheter, the scaffolding expands the graft material to fill the lumen and exerts radial force against the lumen wall.

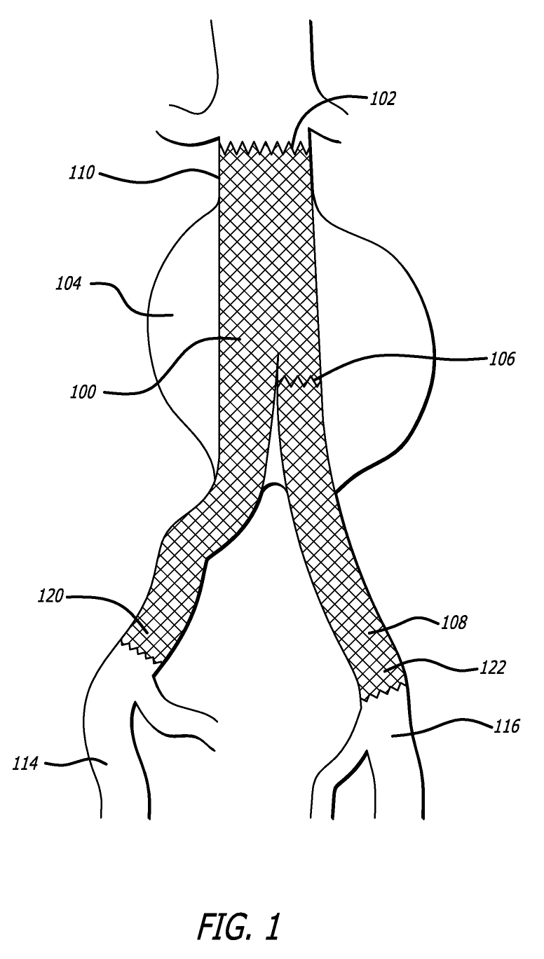

FIG. 1 depicts an exemplary stent graft placement at the site of an abdominal aortic aneurysm. In this type of placement, stent graft 100 is deployed through left iliac artery 114 to aneurysm site 104. Stent graft 100 has distal end 102 and iliac leg 108 to anchor the stent graft in right iliac artery 116. Stent graft 100 is deployed first in a first deployment catheter and iliac leg 108 is deployed in a second deployment catheter and the two segments are joined at overlap 106. Furthermore, after deployment, stent graft 100 contacts the blood vessel wall at least at sites 110, 120 and 122 to prevent leakage of blood into the aneurysm sac at these points.

While stent grafting such as that depicted in FIG. 1 can reduce the possibility of aneurysm rupture, it does not treat the aneurysm itself. That is, even though bypassed and insulated, the aneurysm and its associated diseased tissue remains. The aneurysmic tissue then can continue to degenerate such that the aneurysm continues to increase in size due to the continued thinning of the vessel wall. Thus, methods to treat the diseased tissue in addition to (or in place of) stent grafting would provide a significant advancement in the treatment of aneurysms.

The breakdown of cellular connective tissue such as that along blood vessel walls is a normal physiological process. In healthy vessels, the breakdown of cellular connective tissue exists in a dynamic equilibrium with its re-synthesis and repair. Generally, the MMPs are tightly regulated at the level of their synthesis and secretion and also at the level of their extracellular activity to maintain the appropriate equilibrium with other re-synthesis and repair processes. Over-expression of MMPs or an imbalance between MMPs, however, can lead to excessive tissue breakdown and resulting degenerative disease processes, including but not limited to, aneurysms that are characterized by the excessive breakdown of the extracellular matrix or connective tissues. Thus, inhibiting the actions of MMPs could provide an effective strategy to treat defective vessel walls at aneurysm sites.

Without wishing to be bound by theory, in one aspect, aortic aneurysms can be characterized by distraction of extracellular matrix caused by one or inflammatory processes. Inflammatory cells migrate to atheroscloertic plaque containing areas in a place such as the abdominal aorta. There they are believed to secrete MMPs. This can lead to extracellular matrix degradation. Inflammation requires migration of circulating leukocytes from blood stream to the tissue. This is coordinated by a multistep process of leukocyte transmigration. Vascular endothelial (VE) cadherin is believed to play a key role. VE cadherin is a molecule in inter-endothelial junctions. This is the gate which allows or restricts leukocyte transmigration. Fibrin can bind to VE cadherin and induce an inflammatory process. Moreover, fibrin can orchestrate its own fragmentation. These fragments may be derived from the N-terminal segments of fibrin. These are called E-fragments. Certain fragments may bind VE cadherin and induce inflammation and leukocyte transmigration. Fibrin-derived peptide B-beta (in one embodiment fibrin-derived B-beta 15-42) may inhibit this process and thus treat or stabilize an aneurysm such as an abdominal aneurysm.

Commonly, treatments for various diseases employing MMP inhibition have utilized systemic MMP inhibitors, that is, the MMP inhibitor has been administered either orally, intramuscularly or intra-venously in a dosage sufficient to ensure that the quantity of inhibitor reaching the target site was sufficient to have an effect. One aspect of the present invention is to administer one or more fibrin derived peptide B-beta locally to an aneurysm site utilizing stent grafting procedures. The dispersion of the at least one fibrin derived peptide B-beta allows the therapeutic reaction to be substantially localized so that overall dosages to the individual can be reduced, and undesirable side effects minimized.

At least one fibrin derived peptide B-beta can be delivered to an aneurysm site in three main ways according to the present invention: (1) at least one fibrin derived peptide B-beta can be placed directly onto a stent graft or incorporated into a coating found on a stent graft; (2) at least one fibrin derived peptide B-beta can be provided through a delivery device that is associated with the stent graft, in some embodiments, in association with a carrier and/or (3) at least one fibrin derived peptide B-beta can be administered to the aneurysm site through delivery and/or injection catheters at or near the time of stent graft deployment.

At least one fibrin derived peptide B-beta can be applied to the surface of a stent graft. Following stent graft deployment, the at least one fibrin derived peptide B-beta will diffuse off of the stent graft material to the aneurysm treatment site. When this embodiment is used, at least one fibrin derived peptide B-beta can be applied to the surface of the stent graft using methods including, but not limited to, precipitation, coacervation or crystallization. The at least one fibrin derived peptide B-beta can also be bound to the stent graft covalently, ionically, or through other intramolecular interactions including, without limitation, hydrogen bonding and van der Waals forces.

At least one fibrin derived peptide B-beta can also be incorporated into a coating placed onto the stent graft. Thus, a stent graft coating is a material placed onto the fabric of a stent graft that can hold and release at least one fibrin derived peptide B-beta.

Stent graft coatings used in accordance with the present invention can be either biodegradable or non-biodegradable. Non-limiting representative examples of materials that can be used to produce biodegradable coatings include, without limitation, albumin; collagen; gelatin; fibrinogen; hyaluronic acid; starch; cellulose and cellulose derivatives (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate); casein; dextran; polysaccharides; poly(lactic acid); poly(D,L-lactide); poly(D,L-lactide-co-glycolide); poly(glycolide); poly(hydroxybutyrate); poly(alkylcarbonate); polyesters; poly(orthoesters); poly(ester amide)s (e.g., based on 1,4-butanediol, adipic acid, and 1,6-aminohexanoic acid (BAK 1095)); poly(ester carbonate)s (e.g., tyrosine-poly(alkylene oxide)-derived poly(ether carbonate)s; poly(hydroxyvaleric acid); polydioxanone; poly(malic acid); poly(tartronic acid); polyanhydrides (e.g., poly(adipic anhydride) and poly(sebacic acid-co-1,3-bis(p-carboxyphenoxy)propane)); polyphosphazenes; poly(amino acids); poly(trimethylene carbonate); poly(hydroxy valerate); poly(hydroxy butyrate-co-hydroxy valerate); poly(butylene succinate) (e.g., Bionolle®); poly(butylene adipate); polyarylates (e.g., tyrosine-derived polyarylates); poly(butylene terephthalate)-poly(ethylene glycol) copolymers (polyActive®); poly(\acute{ε}-caprolactone)-b-poly(ethylene glycol)) block copolymers; and poly(ethylene oxide)-b-poly(hydroxy butyrate) block copolymers.

Non-limiting representative examples of materials that can be used to produce non-biodegradable coatings include poly(ethylene-vinyl acetate) (“EVA”) copolymers; silicone rubbers; acrylic polymers (e.g., polyacrylic acid, polymethylacrylic acid, polymethylmethacrylate, polyalkylcynoacrylate); polyethylene; polypropylene; polyamides (nylon 6,6); polyurethane; poly(ester urethanes); poly(ether urethanes); poly(ester-urea); polyethers (e.g., based on poly(oxyethylene) and poly(oxypropylene) units (Pluronic®)); poly(ethylene oxide); poly(propylene oxide); other pluronics; poly(tetramethylene glycol)); and vinyl polymers (e.g., polyvinylpyrrolidone, poly(vinyl alcohol), poly(vinyl acetate phthalate and poly(vinylchloride).

Other useful materials that can be used to produce coatings include polymers such as poly (D,L-lactic acid); poly (L-lactic acid); poly (glycolic acid); poly (caprolactone); poly (valerolactone); copolymers of poly (caprolactone) or poly (lactic acid) with a polyethylene glycol (e.g., MePEG); carboxylic polymers; polyacetates; polyacrylamides; polycarbonates; polyvinylbutyrals; polysilanes; polyureas; polyoxides; polystyrenes; polysulfides; polysulfones; polysulfonides; polyvinylhalides; pyrrolidones; cross-linkable acrylic and methacrylic polymers; vinyl acetate polymers; vinyl acetal polymers; epoxy; melamine; phenolic polymers; water-insoluble cellulose ester polymers (e.g., cellulose acetate propionate, cellulose acetate, cellulose acetate butyrate, cellulose nitrate, and mixtures thereof); polyethylene oxide; polyhydroxyacrylate; poly(ethylene terephthalate); xanthan; hydroxypropyl cellulose; vinyllactam; vinyl butyrolactam; vinyl caprolactam; other vinyl compounds having polar pendant groups; acrylate and methacrylate having hydrophilic esterifying groups; hydroxyacrylate; cellulose esters and ethers; ethyl cellulose; hydroxyethyl cellulose; polyacrylate; natural and synthetic elastomers; rubber; acetal; nylon; styrene polybutadiene; acrylic resin; polycarbonate; polyvinylchloride; polyvinylchloride acetate; pectin; sucrose acetate isobutyrate; hydroxyapatite; tricalcium phosphate; silicates (e.g., Bioglass®, montmorillonite, and mica); alginate; poly(acrylic acid); poly-L-lysine; polyethylenimine; poly(allyl amine); fluorinated polyolefins (e.g., polytetrafluorethylene (Teflon®)); poly(N-isopropylacrylamide); polyacetals; aromatic polyesters; poly(propylene terephthalate) (Sorona®); poly(ether ether ketone)s; and poly(ester imide)s. In general, see U.S. Pat. No. 6,514,515 to Williams; U.S. Pat. No. 6,506,410 to Park, et al.; U.S. Pat. No. 6,531,154 to Mathiowitz, et al.; U.S. Pat. No. 6,344,035 to Chudzik, et al.; U.S. Pat. No. 6,376,742 to Zdrahala, et al.; Griffith, L. A., Ann. N.Y. Acad. of Sciences, 961:83-95 (2002); and Chaikof, et al, Ann. N.Y. Acad. of Sciences, 961:96-105 (2002) the entire contents of each of which are incorporated by reference herein. Additionally, all materials described herein can be blended or copolymerized in various compositions as appropriate, beneficial or required. Such blending or copolymerization is within the level of the ordinary skill in the art.

The selected material used in a particular coating can be obtained from various chemical companies known to those of ordinary skill in the art. However, when polymers are selected as a coating material, because of the potential presence of unreacted monomers, low molecular weight oligomers, catalysts, or other impurities in such commercially available polymers, it can be desirable (or, depending upon the materials used, necessary) to increase the purity of the selected polymer. Such a purification process yields polymers of better-known, purer composition, and therefore increases both the predictability and performance of the mechanical characteristics of the coatings. The exact purification process will depend on the polymer or polymers chosen. Generally, however, in a purification process, the polymer will be dissolved in a suitable solvent. Suitable solvents include (but are not limited to) methylene chloride, ethyl acetate, chloroform, ethanol, and tetrahydrofuran (THF). The polymer solution usually is then mixed with a second material that is miscible with the solvent, but in which the polymer is not soluble, so that the polymer (but not appreciable quantities of impurities or unreacted monomer) precipitates out of solution. For example, a methylene chloride solution of the polymer can be mixed with heptane, causing the polymer to fall out of solution. The solvent mixture then is removed from the copolymer precipitate using conventional techniques.

The coatings used in accordance with the present invention can be fashioned in a variety of forms with desired release characteristics and/or with other specific desired properties. For example, the coatings can be fashioned to release the at least one fibrin derived peptide B-beta upon exposure to a specific triggering event such as increased or decreased pH. Non-limiting representative examples of pH-sensitive coating materials include poly(acrylic acid) and its derivatives (e.g., homopolymers such as poly(aminocarboxylic acid); poly(acrylic acid); poly(methyl acrylic acid); copolymers of such homopolymers; and copolymers of poly(acrylic acid) and other acrylmonomers. Other pH sensitive polymers include polysaccharides such as cellulose acetate phthalate; hydroxypropylmethylcellulose phthalate; hydroxypropyl methylcellulose acetate succinate; cellulose acetate trimellilate; and chitosan. Yet other pH sensitive polymers include any mixture of a pH sensitive polymer and a water-soluble polymer.

Temperature-sensitive polymeric coatings wherein the release of the active agent is dependent on the temperature of the polymer can also be used. Non-limiting representative examples of temperature-sensitive materials and their gelatin temperature include homopolymers such as poly(N-methyl-N-n-propylacrylamide) (19.8° C.); poly(N-n-propylacrylamide) (21.5° C.); poly(N-methyl-N-isopropylacrylamide) (22.3° C.); poly(N-n-propylmethacrylamide (28.0° C.); poly(N-isopropylacrylamide) (30.9° C.); poly(N,n-diethylacrylamide) (32.0° C.); poly(N-isopropylmethacrylamide) (44.0° C.); poly(N-cyclopropylacrylamide) (45.5° C.); poly(N-ethylmethyacrylamide) (50.0° C.); poly(N-methyl-N-ethylacrylamide) (56.0° C.); poly(N-cyclopropylmethacrylamide) (59.0° C.); and poly(N-ethylacrylamide) (72.0° C.). Cellulose ether derivatives such as hydroxypropyl cellulose (41° C.); methyl cellulose (55° C.); hydroxypropylmethyl cellulose (66° C.); and ethylhydroxyethyl cellulose as well as pluronics such as F-127 (10-15° C.); L-122 (19° C.); L-92 (26° C.); L-81 (20° C.); and L-61 (24° C.) can also be used. Moreover, temperature-sensitive materials can be made by preparing copolymers between (among) monomers of the above, or by combining such homopolymers with other water-soluble polymers such as acrylmonomers (e.g., acrylic acid and derivatives thereof such as methylacrylic acid, acrylate and derivatives thereof such as butyl methacrylate, acrylamide, and N-n-butyl acrylamide).

Coatings used in accordance with the present invention can also be prepared in a variety of paste or gel forms. For example, within one embodiment of the invention, coatings are provided which are liquid at one temperature (e.g., a temperature greater than about 37° C., such as about 40° C., about 45° C., about 50° C., about 55° C. or about 60° C.), and solid or semi-solid at another temperature (e.g., ambient body temperature, or any temperature lower than about 37° C.). As is understood by one of ordinary skill in the art, such pastes or gels can be made utilizing a variety of techniques. Other pastes or gels can be applied as a liquid, which can solidify in vivo due to dissolution of a water-soluble component of the paste and precipitation of encapsulated drug into the aqueous body environment.

Coatings can be fashioned in any appropriate thickness. For example, coatings can be less than about 2 mm thick, less than about 1 mm thick, less than about 0.75 mm thick, less than about 0.5 mm thick, less than about 0.25 mm thick, less than about 0.10 mm thick, less than about 50 μm thick, less than about 25 μm thick or less than about 10 μm thick. Generally, such coatings will be flexible with a good tensile strength (e.g., greater than about 50, greater than about 100, or greater than about 150 or 200 N/cm²), have good adhesive properties (i.e., adhere to moist or wet surfaces), and have controlled permeability.

As is understood by one of ordinary skill in the art, at least one fibrin derived peptide B-beta can be, without limitation, linked by occlusion in the matrices of a coating, bound by covalent linkages, to the coating or medical device itself or encapsulated in microcapsules within the coating. Within certain embodiments, the at least one fibrin derived peptide B-beta can be provided in noncapsular formulations such as, without limitation, microspheres (ranging from nanometers to micrometers in size), pastes, threads of various size, films or sprays.

Coatings used in accordance with the present invention can be formulated to deliver the at least one fibrin derived peptide B-beta over a period of about several minutes, several hours, several days, several months or several years. For example, “quick release” or “burst” coatings can release greater than about 10%; greater than about 20%, or greater than about 25% (w/v) of the at least one fibrin derived peptide B-beta over a period of about 7 to about 10 days. “Slow release” coatings can release less than about 1% (w/v) of the at least one fibrin derived peptide B-beta over a period of about 7 to about 10 days. “Medium-release” coatings can have release profiles between the quick-release and slow-release profiles.

In one embodiment, coatings used in accordance with the present invention can be coated with a physical barrier to protect the coating during packaging, storage and deployment procedures Physical barriers can also be used to affect the release profile of at least one fibrin derived peptide B-beta from the coating once the stent graft is deployed. Such barriers can include, without limitation, inert biodegradable materials such as gelatin, poly(lactic-co-glycolic acid)/methoxypolyethyleneglycol film, polylactic acid, or polyethylene glycol. In the case of poly(lactic-co-glycolic acid)/methoxypolyethyleneglycol, once the poly(lactic-co-glycolic acid)/methoxypolyethyleneglycol becomes exposed to blood, the methoxypolyethyleneglycol will dissolve out of the poly(lactic-co-glycolic acid), leaving channels through the poly(lactic-co-glycolic acid) to the underlying coating containing at least one fibrin derived peptide B-beta.

Protection of the coating and its at least one fibrin derived peptide B-beta also can be achieved by covering the coating's surface with an inert molecule that prevents access to the coating and at least one fibrin derived peptide B-beta through steric hindrance. The coating can also be covered with an inactive form of at least one fibrin derived peptide B-beta, which can later be activated. For example, in one embodiment the coating could be coated with an enzyme, which causes either the release of the at least one fibrin derived peptide B-beta or activates the at least one fibrin derived peptide B-beta. Activation can also be achieved by injecting another material into the aneurysm sac after the stent graft is deployed.

Another example of a suitable physical barrier over the coating is an anti-coagulant (e.g., heparin), which can be applied over the top of the at least one fibrin derived peptide B-beta -containing coating. The presence of an anti-coagulant can delay coagulation. As the anticoagulant dissolves away, the anticoagulant activity stops, and the newly exposed at least one fibrin derived peptide B-beta coating can initiate its intended action.

In some embodiments, alternating layers of the at least one fibrin derived peptide B-beta coating with a protective coating can enhance the time-release properties of the coating overall.

Coatings according to the present invention can be applied according to any technique known to those of ordinary skill in the art of medical device manufacturing. For example, coatings can be applied to the stent grafts used in accordance with the present invention as a “spray”, which solidifies into a coating. Such sprays can be prepared from microspheres of a wide array of sizes, including for example and without limitation, from about 0.1 μm to about 3 μm, from about 10 μm to about 30 μm or from about 30 μm to about 100 μm. Additionally or alternatively, coatings can be applied by, without limitation, impregnation, spraying, brushing, dipping and/or rolling. In another embodiment, a polymer- at least one fibrin derived peptide B-beta blend can be used to fabricate fibers or strands that are embedded within the fabric of the stent graft. After a coating is applied, it can be dried. Drying techniques include, but are not limited to, heated forced air, cooled forced air, vacuum drying or static evaporation.

For additional information regarding stents, stent grafts and coatings, see U.S. Pat. No. 6,387,121 to Alt; U.S. Pat. No. 6,451,373 to Hossainy, et al.; and U.S. Pat. No. 6,364,903 to Tseng, et al the entire contents of each of which are incorporated by reference herein.

In place of or in addition to coatings on a stent graft, at least one fibrin derived peptide B-beta can also be administered to an aneurysm site following stent graft deployment with the use of a delivery device associated with the stent graft. In such embodiments, the stent graft isolates the aneurysm site from blood flow and provides a structure to which the delivery device can be attached. In this manner, at least one fibrin derived peptide B-beta can be delivered directly to the aneurysm site and not to surrounding healthy tissue. The at least one fibrin derived peptide B-beta are released into this relatively sealed environment such that they are largely limited to this region. Thus, a maximum concentration of the at least one fibrin derived peptide B-beta remains at the treatment site and is not delivered to the rest of the body. As a result, substantial quantities of the at least one fibrin derived peptide B-beta remain at the treatment site for a longer period of time, increasing the efficacy of the at least one fibrin derived peptide B-beta potential.

Delivery devices, as described herein, can include, without limitation, a pouch that is attached to the stent graft or made from stent graft layers wherein the at least one fibrin derived peptide B-beta (and associated carriers when used) are placed inside the pouch.

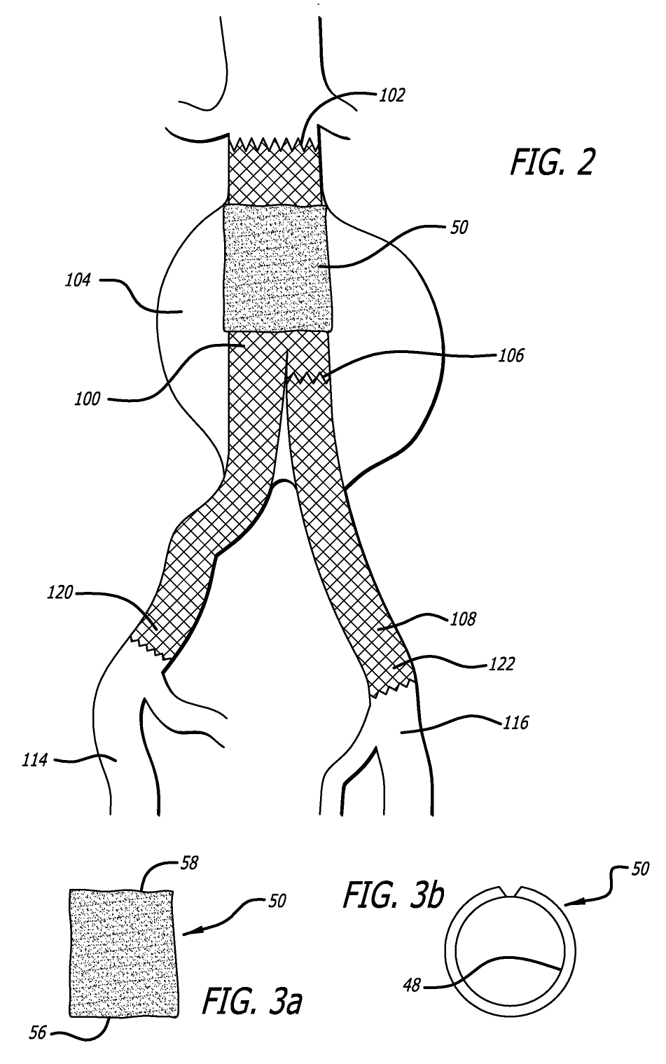

FIG. 2 depicts an at least one fibrin derived peptide B-beta delivery device in the form of pouch 50. In this exemplary embodiment, pouch 50 is connected to ring 48 on the outer surface of stent graft 22. Delivery device 50 is positioned such that upon placement at an aneurysm site (in the depicted example, aneurysmal sac 18 of aorta 10), delivery device 50 is located between stent graft 22 and aneurysmal wall 16 of aorta 10.

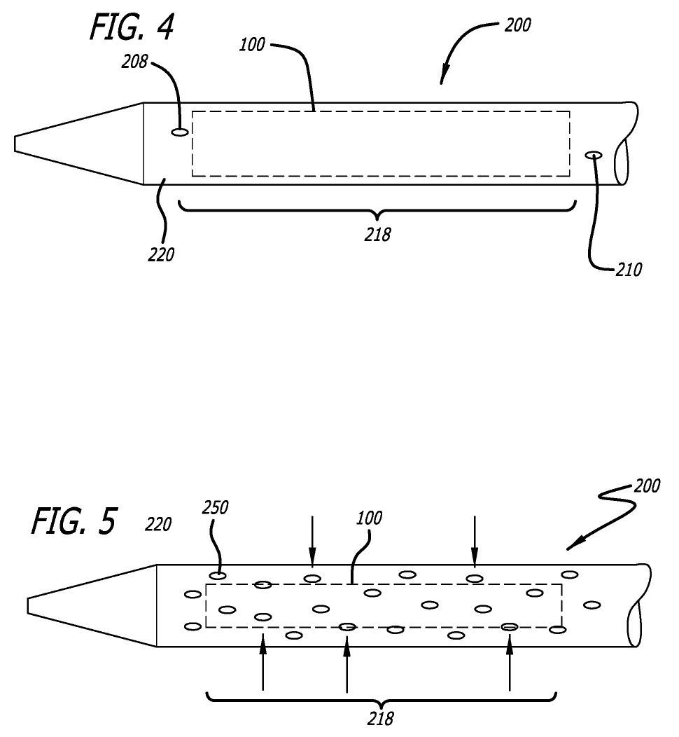

FIG. 3a depicts pouch 50. Pouch 50 can be wrapped around the outer wall of the stent graft and attached, in one embodiment, at end 58 of pouch 50. Pouch 50 can be prepared, for example, by folding a sheet of the pouch material in half, and attaching together the opposed sides projecting from the crease occurring at the fold which forms end 56, such as by sewing, laser welding, adhesives or the like to leave an open end. The at least one fibrin derived peptide B-beta (with or without carriers) are then loaded into the interior of the pouch 50. Open end 58 can then be sealed. FIG. 3b shows a top cross-sectional view of pouch 50 attached to ring 48 of stent graft 22.

Alternatively, multiple pouches can be used, with each pouch being attached to the stent graft. In one embodiment the pouches are arranged so that the spacing between adjacent pouches extending about the circumference of the stent graft is relatively equal. In one embodiment, at least four such delivery devices are equally spaced about the circumference of the stent graft. Alternatively, multiple delivery devices can be located both about the circumference of the stent graft, as well as longitudinally along the stent graft. In another embodiment, appropriately placed pouches can be created by adopting a stent graft that includes two fabric layers. The fabric layers can be adhered together at various places to create any desired number or configuration of pouches.

When used with the described delivery devices, at least one fibrin derived peptide B-beta carriers can be, without limitation, a sheet, a slab, a gel, a capsule or capsules, microparticles, nanoparticles and/or combinations of these. For example, a carrier could comprise a polymeric sheet loaded with at least one fibrin derived peptide B-beta. Such a sheet can be formed by dissolving or dispersing both the polymer and at least one fibrin derived peptide B-beta in a suitable solvent, pouring this solution into a suitable mold and removing the solvent by evaporation. The formed sheet can then be cut to fit the delivery device.

Alternatively, a gel can be used as a carrier for at least one fibrin derived peptide B-beta. Such a gel can be prepared by dissolving a polymer in an organic solvent in which the at least one fibrin derived peptide B-beta are either dissolved or dispersed. The gel can be placed into the delivery device, and when the stent graft is implanted, release at least one fibrin derived peptide B-beta into the aneurysmal sac, where the delivery device provides a convenient mechanism to maintain the gel adjacent the aneurysmal sac.

As with coatings described above, the delivery device and/or carrier can be biodegradable or non-biodegradable and fashioned with any of the materials described above. As such, the same desired release characteristics and properties can be achieved including those described above relating to ph or temperature sensitivity, quick, medium or slow release profiles, physical barriers, etc.

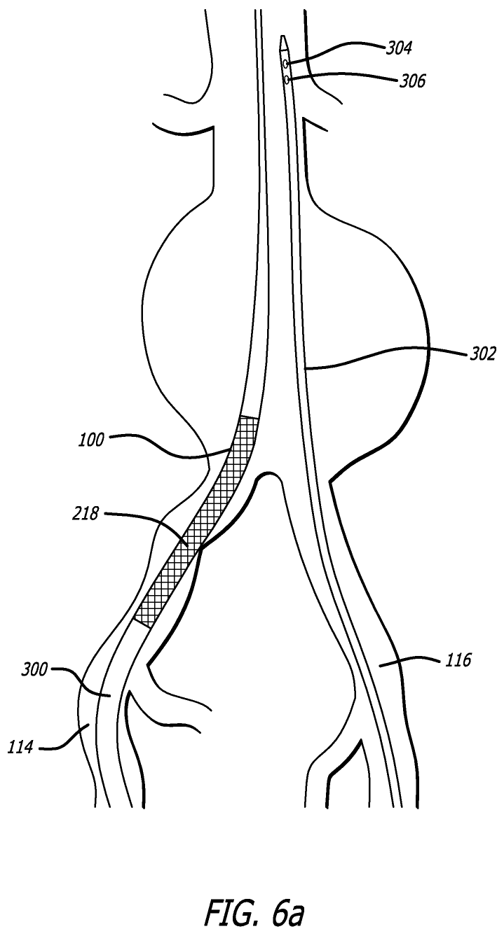

At least one fibrin derived peptide B-beta can also be delivered to the site of an aneurysm using delivery and/or injection catheters at or near the time of stent graft deployment. In one embodiment, a stent graft is pre-loaded into a delivery catheter such as that depicted in FIG. 4. Stent graft 100 is radially compressed to fill stent graft chamber 218 in the distal end of delivery catheter 200. Stent graft 100 is covered with retractable sheath 220. In this depicted embodiment, delivery catheter 200 has first injection port 208 and second injection port 210 for applying at least one fibrin derived peptide B-beta onto the outer wall of the stent graft prior to deployment. Stent graft 100 is then deployed to the treatment site as depicted in FIG. 1.

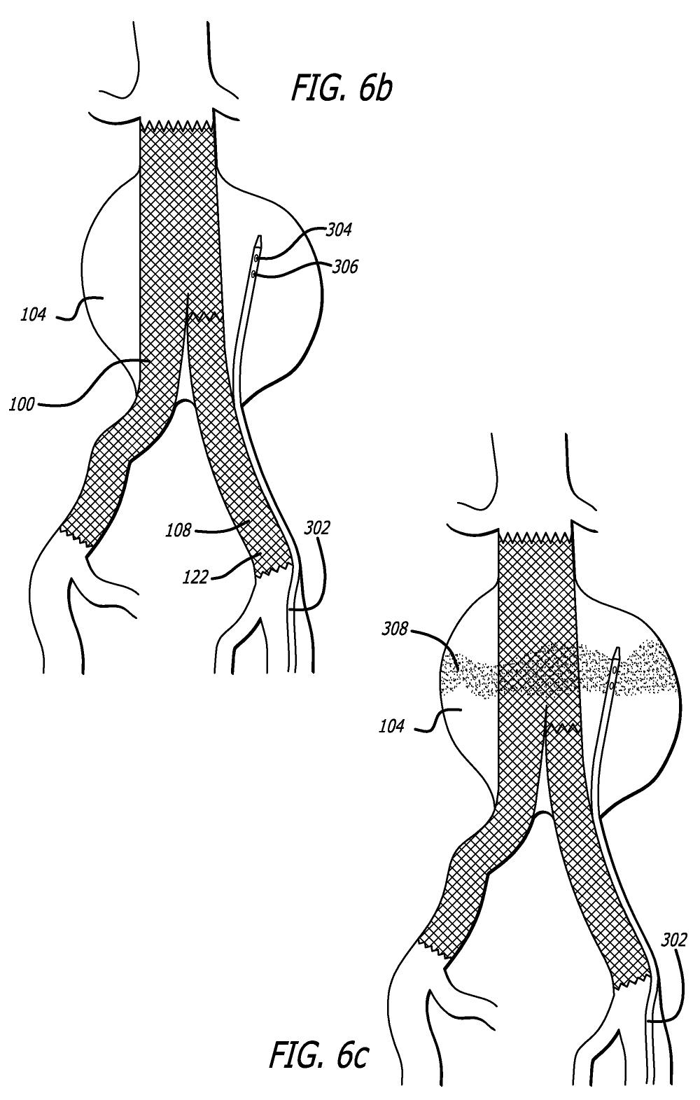

Another embodiment for coating the outer wall of stent graft 100 within delivery catheter 200 is depicted in FIG. 5. Retractable sheath 220 contains plurality of holes 250 through which at least one fibrin derived peptide B-beta can be applied to the outer wall of stent graft 100 compressed within stent graft chamber 218 prior to deployment. Stent graft 100 is then deployed to the treatment site as depicted in FIG. 1.

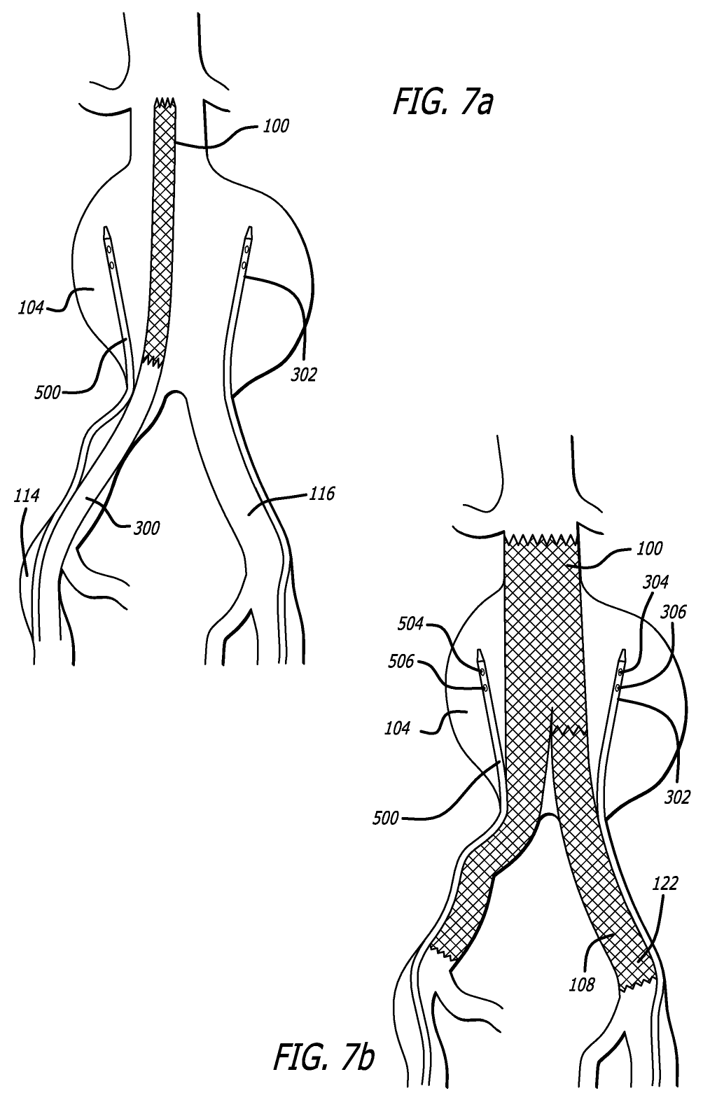

In another embodiment, at least one fibrin derived peptide B-beta are injected between the stent graft and the vessel wall during or after stent graft placement. As depicted in FIG. 6a, stent graft 100 is radially compressed to fill stent graft chamber 218 of stent delivery catheter 300 which is then deployed to the treatment site via left iliac artery 114. Multilumen injection catheter 302 is also deployed to the treatment site through right iliac artery 116. Multilumen injection catheter 302 can be a coaxial catheter with two injection lumens or a dual lumen catheter or alternatively a three lumen catheter if a guide wire lumen is required. Injection catheter 302 has first injection port 304 and second injection port 306 through which at least one fibrin derived peptide B-beta can be delivered to a treatment site. In the first step of this deployment scheme (FIG. 6a), stent delivery catheter 300 and injection catheter 302 are deployed independently to the treatment site.

FIG. 6b shows stent graft 100 deployed. In this depicted embodiment, delivery catheter 300 has been removed and iliac limb 108 has been deployed. Iliac limb segment 108 of stent graft 100 seals the aneurysm sac at proximal end 122. Injection catheter 302 has also been retracted so that first injection port 304 and second injection port 306 are within aneurysmal sac 104. At least one fibrin derived peptide B-beta 308 can then be injected between the vessel lumen wall and the stent graft within aneurysm sac 104 (FIG. 6c). Injection catheter 302 is then retrieved.

In another embodiment, a single lumen injection catheter can be used in the place of a multilumen injection catheter. After the guide wire is retrieved from the lumen, at least one fibrin derived peptide B-beta can be delivered to the treatment site through the same lumen of the single lumen injection catheter. In an alternate embodiment, more than one single lumen injection catheter can be deployed in each iliac artery with the distal ends of the catheters meeting in the aneurysm sac.

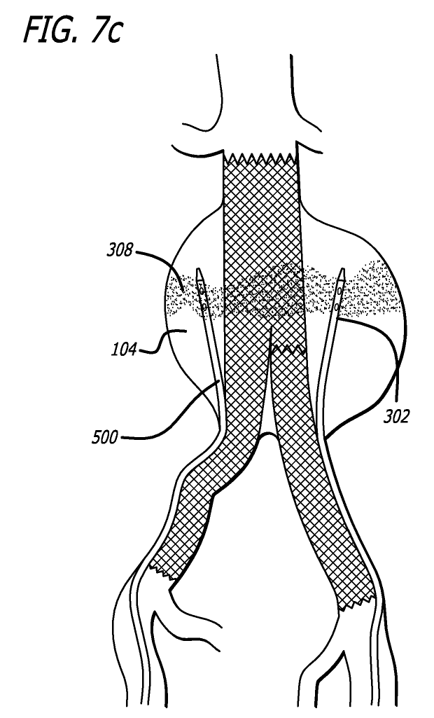

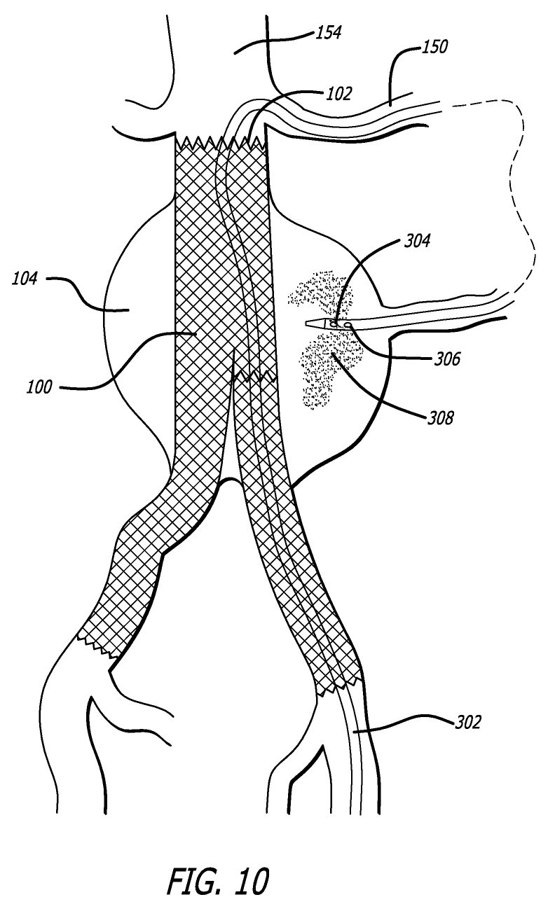

In another alternative embodiment, more than one injection catheter can be used to deliver at least one fibrin derived peptide B-beta to the aneurysm sac (FIG. 7a). As previously described in FIGS. 1 and 6, stent graft 100 is deployed to the treatment site via left iliac artery 114 (FIG. 7a). Multiple single lumen or multilumen injection catheters 302 and 500 are also deployed to aneurysm sac 104 through right iliac artery 116 and left iliac artery 114 (FIG. 10a). Injection catheters 302 and 500 have injection ports through which at least one fibrin derived peptide B-beta can be deposited. Delivery catheter 300 is removed with both stent graft limbs deployed as in FIG. 7b while injection catheters 302 and 500 remain in place with injection ports 304 and 306 and 504 and 506 in aneurysm sac 104. Iliac limb segment 108 of stent graft 100 seals the aneurysm sac at the proximal end 122. At least one fibrin derived peptide B-beta 308 are then administered to aneurysm sac 104 (FIG. 7c) and injection catheters 302 and 500 can then be retrieved.

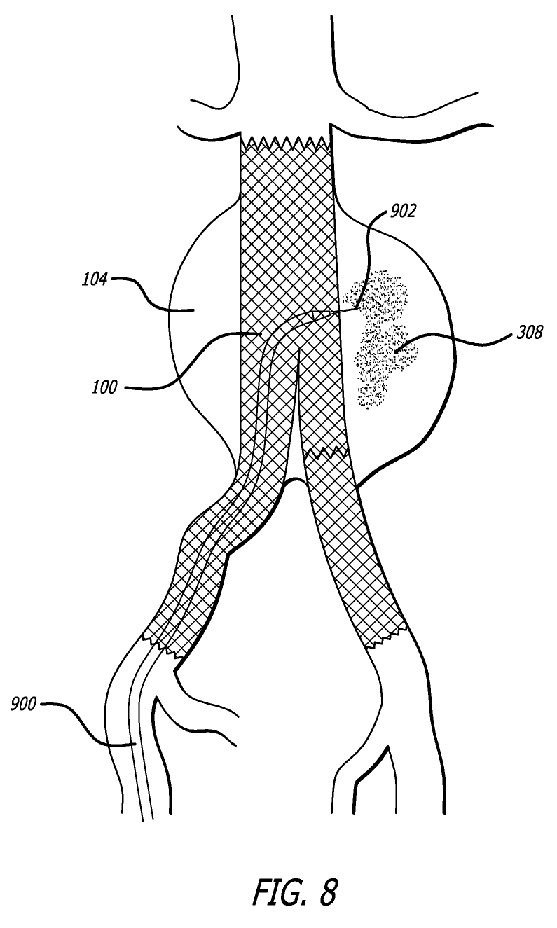

In yet another embodiment, at least one fibrin derived peptide B-beta can be delivered to aneurysm sac 104 by injecting the components through the wall of stent graft 100 (FIG. 8). Injection catheter 900 is advanced to the site of an already deployed stent graft 100 and needle 902 penetrates stent graft 100 to deliver at least one fibrin derived peptide B-beta 308 to aneurysm sac 104. Injection catheter 900 can be a multi-lumen or single lumen catheter.

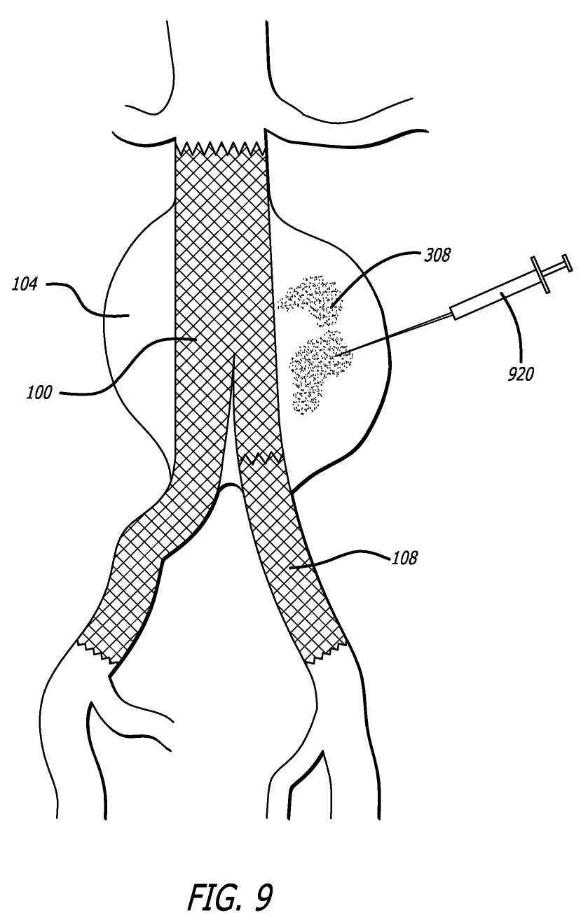

In another embodiment, at least one fibrin derived peptide B-beta are delivered to aneurysm sac 104 by translumbar injection (FIG. 9). Injection device 920, such as but not limited to a syringe, is directed, under radiographic or echographic guidance, to the aneurysm sac where stent graft 100 and iliac leg 108 have already been deployed. Injection device 920 delivers which at least one fibrin derived peptide B-beta 308 to aneurysm sac 104. Injection device 920 can have a single lumen or multiple lumens.

In yet another embodiment, depending on aneurysm location and stent graft placement, a collateral artery can be used to access the aneurysm sac (FIG. 10). For example, and not intended as a limitation, stent graft 100 can be deployed such that distal end 102 is in abdominal aorta 154 near, but below the renal artery. After deployment of stent graft 100, the deployment catheter is removed and injection catheter 302 is advanced up the aorta past aneurysm sac 104 to superior mesenteric artery 150. Injection catheter 302 is then advanced through superior mesenteric artery 150 and down into the inferior mesenteric artery where it originates at the aorta within aneurysm sac 104. At least one fibrin derived peptide B-beta 308 can then be injected into aneurysm sac 104 through first injection port 304 and second injection port 306.

In addition to the site specific delivery of at least one fibrin derived peptide B-beta, one or more additional bioactive agent can also be locally administered according to the present invention. The choice of bioactive agent to incorporate, or how much to incorporate, can have a great deal to do with, in one embodiment, a polymer selected to coat the stent graft. A person of ordinary skill in the art appreciates that hydrophobic agents prefer hydrophobic polymers and hydrophilic agents prefer hydrophilic polymers. Therefore, coatings can be designed for agent or agent combinations with immediate release, medium release or slow release profiles.

Non-limiting examples of particular bioactive agents or types of bioactive agents that may be particularly beneficial within the context of the present invention include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, matrix metalloproteinase inhibitors and transforming nucleic acids. Bioactive agents can also include anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like. Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method of treating an aneurysm comprising:
delivering a stent graft to the site of said aneurysm;
deploying said stent graft to span said aneurysm; and
locally administering at least one fibrin-derived peptide B-beta to said site of said aneurysm.

2. The method according to claim 1 wherein said locally administering comprises:
applying said at least one fibrin-derived peptide B-beta to the outer surface of said stent graft and/or
incorporating said at least one fibrin-derived peptide B-beta into a coating on said stent graft.

3. The method according to claim 1 wherein said locally administering comprises:
incorporating said at least one fibrin-derived peptide B-beta into a coating; and
placing said coating on the outer surface of said stent graft.

4. The method according to claim 1 wherein said locally administering comprises:
attaching a delivery device to said stent graft wherein said delivery device holds and releases at least one fibrin-derived peptide B-beta.

5. The method according to claim 4 wherein said delivery device is a pouch.

6. The method according to claim 1 wherein said locally administering comprises:
providing a stent graft with two layers wherein following deployment the first layer is exposed to blood flow and the second layer faces the blood vessel wall and wherein said second layer is semi-permeable;
partially adhering the layers together so that pouches are formed; and
loading said pouches with at least one fibrin-derived peptide B-beta.

7. The method according to claim 6 wherein said method further comprises:
associating said at least one fibrin derived peptide B-beta with a carrier before loading said pouches with said at least one fibrin derived peptide B-beta.

8. The method according to claim 1 wherein said locally administering comprises:
applying at least one fibrin derived peptide B-beta directly to the outer surface of said stent graft while said stent graft is compressed within a stent deployment catheter.

9. The method according to claim 1 wherein said locally administering comprises:
administering said at least one fibrin derived peptide B-beta through a delivery catheter and/or an injection catheter.

10. The method according to claim 9 wherein said at least one fibrin derived peptide B-beta substantially fill the aneurysm sac.

11. The method according to claim 9 wherein said injection catheter is selected from the group comprising a single lumen injection catheter and a multilumen injection catheter.

12. The method according to claim 9 comprising:
administering said at least one fibrin derived peptide B-beta through at least two injection catheters wherein the first and second injection catheters reach said aneurysm through a different route.

13. A stent graft comprising at least one fibrin derived peptide B-beta wherein said at least one fibrin derived peptide B-beta are one or more of applied to the outer surface of said stent graft, incorporated within a coating applied to said stent graft or within a delivery device associated with said stent graft.

14. The stent graft according to claim 13 wherein said stent graft comprises at least one fibrin derived peptide B-beta incorporated within a coating applied to said stent graft wherein said coating is biodegradable.

15. The stent graft according to claim 13 wherein said stent graft comprises at least one fibrin derived peptide B-beta incorporated within a coating applied to said stent graft wherein said coating is temperature-sensitive and/or pH-sensitive.

16. The stent graft according to claim 13 wherein said stent graft comprises at least one fibrin derived peptide B-beta incorporated within a coating applied to said stent graft wherein said coating is formulated to be a quick-release coating, a medium-release coating or a slow-release coating.

17. The stent graft according to claim 13 wherein said stent graft comprises at least one fibrin derived peptide B-beta within a delivery device associated with said stent graft and wherein said at least one fibrin derived peptide B-beta are further associated with a carrier.

18. The stent graft according to claim 17 wherein said carrier is selected from the group consisting of a sheet, a slab, a gel, a capsule, capsules, microparticles, nanoparticles, and combinations thereof.

19. The stent graft according to claim 13 wherein said delivery device is a pouch associated with said stent graft.

20. The stent graft according to claim 19 wherein said pouch is created by providing a stent graft with two layers wherein following deployment the first layer is exposed to blood flow and the second layer faces the blood vessel wall and wherein said second layer is semi-permeable; and
partially adhering the layers together so that one or more pouches are formed.
